(19) United States
(12) Patent Application Publication
(10) Pub. No.: US 2007/0168011 A1
(43) Pub. Date: Jul. 19, 2007

(54) DETACHABLE THERAPEUTIC MATERIAL

(75) Inventors: Robert C. LaDuca, Santa Cruz, CA (US); Carol LaSalle, Redwood City, CA (US)

Correspondence Address: LEVINE BAGADE HAN LLP, 2483 EAST BAYSHORE ROAD, SUITE 100, PALO ALTO, CA, 94303, US

(73) Assignee: Duke Fiduciary, LLC, Santa Cruz, CA (US)

(21) Appl. No.: 11/559,858

(22) Filed: Nov. 14, 2006

Related U.S. Application Data
(63) Continuation-in-part of application No. 11/274,623, filed on Nov. 14, 2005.

(51) Int. Cl. A61F 2/84

(52) U.S. Cl. 623/1.11; 606/108

(57) ABSTRACT

The invention provides implantable devices comprising a biocompatible material that provides a structural function or therapeutic function or both. The devices are configured to be detachable or releasable from a distal portion of a delivery instrument such as a catheter.

### CROSS-REFERENCE TO RELATED APPLICATIONS

This application is a continuation-in-part of U.S. patent application Ser. No. 11/274,623, filed Nov. 14, 2005, which is incorporated herein by reference in its entirety.

### BACKGROUND OF INVENTION

In 2003 and 2004, the U.S. Food and Drug Administration approved two different drug-eluting stents for angioplasty procedures to open clogged coronary arteries. A drug-eluting stent is a metal stent that has been coated with a pharmacologic agent that interferes with restenosis, or the reblocking of the artery. Each year close to 1 million angioplasty procedures are performed, and of those some 30% of patients experience restenosis within one year, requiring further treatment such as repeat angioplasty or coronary artery bypass surgery. With the advent of drug eluting stents that elute anti-restenotic drugs, the incidence of restenosis after stent placement has been reduced to single digits.

Effectiveness of the drug-eluting stent depends at least in part on the type of metal stent used, the coating selected and the pharmacological agent selected, how the agent is released at the site, and whether the stent has been properly placed in the artery to prevent the complications of blood clots or sub-acute thrombosis. Early trials using drug-eluting stents indicate that they are much more successful at treating patients than bare stents alone. Currently available stents include a paclitaxel-eluting stent (that releases the chemotherapeutic drug paclitaxel) and a sirolimus-eluting stent (that releases the immunosuppressant simolimus). Both stents are bare metal stents that have been coated with a slow to moderate release drug formulation embedded in a polymer. The drug is selected based on its ability to slow or inhibit the process of restenosis, which is sometimes characterized as epithelial cell hyperplasia in response to the injury of angioplasty or stent placement. Both products have proven successful in clinical trials in comparison with bare metal stents or angioplasty alone. Presently, data from clinical trials indicates a four-fold reduction in the incidence of restenosis with medicated stents.

Because the drugs currently used in the drug-eluting stents delay endothelisation by inhibiting fibroblast proliferation, one side effect of drug-eluting stents is the risk of thrombosis in or about the stent within the 6 months following stent's placement. For this reason, patients implanted with drug-eluting stents receive anti-coagulants, such as clopidogrel or ticlopidine, for up to 6 months following placement of the device to prevent thrombosis. If the system works, a smooth thin layer of endothelial cells (which is the inner lining of the blood vessel) grows over the stent during this period and the device is incorporated into the artery, reducing the tendency for clotting.

It would be advantageous to develop other ways to treat diseased or damaged vessels that overcome the drawbacks of stents and other prior art devices and procedures.

### SUMMARY OF THE INVENTION

The invention provides implantable devices comprising a biocompatible material that provides a structural function or therapeutic function or both. The devices are configured to be detachable or releasable from a distal portion of a delivery instrument such as a catheter.

In many variations, the devices are in the form of a luminal or hollow structure having an exterior surface and an interior surface whereby the exterior surface is configured for engagement against the interior wall of the tissue structure into which the device is to be implanted and the interior surface is configured to contact or be exposed to the interior environment of the tissue structure. In other variations, the device has a planar structure and, as such, can be used as a covering or patch to overlie a target tissue surface or to cover a defect therein where that surface may be an interior or an exterior surface of a vessel, organ or body cavity. Either one or both sides of the device in a planar configuration may be configured for engagement against a tissue structure.

The material from which the implantable devices are fabricated is configured or treated to provide a structural, biological and/or therapeutic effect at the implant site. The device is preferably comprised of material which is at least in part biodegradable or resorbable. Examples of suitable materials include natural or synthetic extracellular matrices as these materials may be constructed to function as a scaffold for buttressing the treatment site, retaining the device at the site, biologically remodeling the site and/or providing a structure to which therapeutic agents may be applied for elution at the implant site. Other components which may be used to form the implantable devices include biodegradable and non-biodegradable stents or stent-like structures to which the biological material may be applied. The devices may further comprise compositions such as elutable therapeutic agents for treating or preventing one or more conditions at the implant site.

The invention further includes systems for the delivery and placement of the subject devices at a target implant site within the body. The systems include an implant delivery instrument, such as a catheter or sheath. The systems may further include a guidewire over which the catheter is translated. Other system components may be employed, such as balloon catheters, inflation mediums, nose coned guidewires, etc., depending on the type of implant used, and whether or not a stent is also used to deploy the implant. As the implant site may be any tubular or hollow tissue lumen or organ, or both, the delivery systems of the present invention may be particularly designed to for percutaneous, endovascular, oral, buccal, parenteral or rectal delivery procedures.

Another feature of the present invention attachment-detachment arrangement between the implantable device and the delivery system. The implants are physically attached or secured in a releasable manner to the delivery system. Suitable mechanical attachment-detachment mechanism include but are not limited to one or a plurality of sutures, strings, magnets, clips, hooks, etc. Another modality of releasable attachment is the use of a bio-adhesive to secure the implant to the delivery system where the adhesive material has properties which enable it to dissipate or dissolve when exposed to moisture and/or body heat at the target tissue site. Another modality for the releasable attachment of the implant to the delivery device is by way of perforations made in the implant material which can be caused to split or tear away from the delivery device when a force is applied to the implant.

The invention further provides a method of making the subject implantable devices where the device includes a biocompatible material having at least two surfaces. The fabrication process may comprises a process selected from the group consisting of extruding, sewing, laminating, pressing, freeze-drying, gluing, and molding the material to provide the desired shape and construct. Fabrication also includes treating or configuring the material as desired or necessary to provide the desired structural support retain the device once implanted and/or to induce the desired biological and/or therapeutic effect at the implant site. The fabrication methods also include providing releasable attachment of the implant to the delivery system to be used. In one embodiment, this involves the formation of perforations within the material.

The invention further provides various methods of treating a target tissue site where the treatment process one or more of buttressing the tissue site, forming healthy new tissue at the tissue site, and eluting a bioactive agent or drug at the target site.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Also for purposes of clarity, certain features of the invention may not be depicted in some of the drawings. Included in the drawings are the following figures:

FIGS. 1A-1D illustrates various configurations of the implantable devices of the present invention; namely, tubular, global, planar and tubular-planar configurations, respectively.

FIGS. 2A-2C illustrate cross-sectional views of tubular implants which are folded, pleated and rolled, respectively, into a reduced diameter state.

FIGS. 3A and 3B illustrate cross-sectional views of planar implants which are folded and rolled, respectively, into a reduced profile state.

FIGS. 4A and 4B illustrate alternative locations at which the implant may be secured to a delivery device of the present invention.

FIGS. 5A-5C illustrate implants having alternative numbers and locations of perforations for release of one or more portions of the implants.

FIGS. 6A and 6B illustrate an implantable device in operative use with a delivery-placement system of the present invention which employs a balloon-expandable stent for deploying and seating the implant at a target site.

FIGS. 7A and 7B illustrate an implantable device in operative use with another delivery-placement system of the present invention which employs a guide wire having a nose cone at its distal end to facilitate delivery of the system and deployment of the implant at a target site.

### DETAILED DESCRIPTION OF THE INVENTION

Before the devices, systems and methods of the present invention are described, it is to be understood that this invention is not limited to particular therapeutic applications and implant sites described, as such may vary. It is also to be understood that the terminology used herein is for the purpose of describing particular embodiments only, and is not intended to be limiting, since the scope of the present invention will be limited only by the appended claims. Unless defined otherwise, all technical and scientific terms used herein have the same meaning as commonly understood by one of ordinary skill in the art to which this invention belongs.

The terms “proximal” and “distal”, when used with respect to the implant delivery and placement systems of the present invention, are to be understood to indicate positions or locations relative to the user where proximal refers to a position or location closer to the user and distal refers to a position or location farther away from the user. When used with reference to the implantable devices of the present invention, these terms are to be understood to indicate positions or locations relative to a delivery and placement system when the implantable devices is operatively engaged with or positioned in the vicinity of the system. As such, proximal refers to a position or location closer to the proximal end of the delivery and placement system and distal refers to a position or location closer to the distal end of the delivery and placement system.

The term “implant” or “implantable device” as used herein includes but is not limited to a device comprising a material having any suitable structure, shape or flexibility/stiffness to optimally seat and engage within/on a vessel, organ or other tissue structure into/onto which it is to be implanted. The implant or device may further include other structures, materials, coatings, agents or the like or combinations thereof, which perform a therapeutic or other function (e.g., facilitating visualization of the implant, stabilizing or securing the positioning of the implant within the implant site, lubricating the implant to facilitate the delivery, etc.).

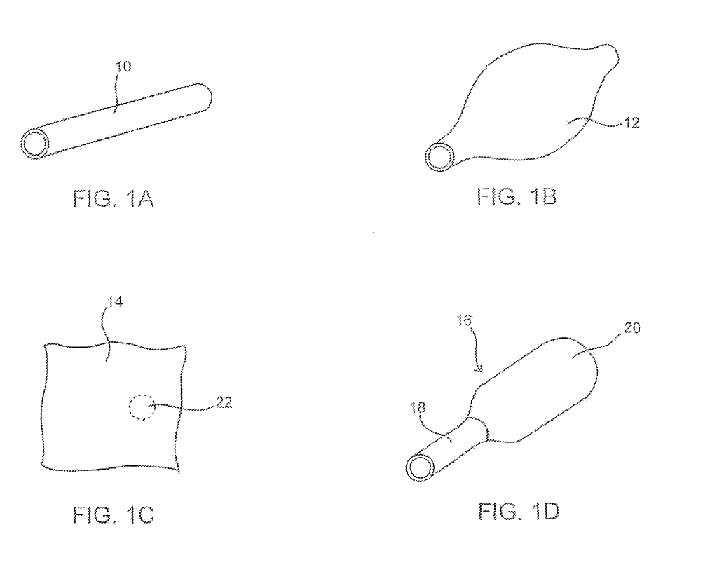

The shape, size and dimensions of an implant are dictated substantially by those of the tissue site into/onto which it is to be implanted. In one variation, as illustrated in FIG. 1A, the implant 10 has an open tubular structure which is suitable for placement within tubular-shaped tissue structures or organs through which fluids or other materials are passed. Examples of applicable tissue structures include blood vessels (including but not limited to coronary vessels, carotid vessels, intracranial vessels, peripheral vessels, adjacent aneurysms, arteriovenous malformations, arteriovenous fistulas, etc.), portions of the intestinal tract, urethra, fallopian tubes, ducts such as bile ducts and mammary ducts, large and small airways, etc. In other variations, the implant, while hollow, may have less of a tubular structure and more of a globe-like or other amorphous or voluminous structure to address non-tubular organs or other tissue structures, e.g., stomach, intestines, kidneys, bladder, colon, a cardiac chamber, etc. An example of a device 12 having global configuration is illustrated in FIG. 1B. Planar variations of the device, such as implant 14 of FIG. 1C, are also provided whereby they are configured to cover an interior or exterior surface of a tissue structure where the tissue structure may itself have a tubular, hollow or planar surface or configuration which requires treatment by the implant. Applicable tissue structures include, for example, the endocardium, epicardium, myocardium, liver, lungs, stomach lining, pancreas, the mouth of an aneurysm, and for hernia repair applications. Still yet, the implants may be provided which have a more customized construct in which one or more planar portions and one or more luminal portions are employed together to address, for example, organ to vessel junctures. An example of such a device 16 having a tubular portion 18 and a planar portion 20 is illustrated in FIG. 1D.

The subject implants may have one or more openings or aspects to be aligned with target features of the resident anatomy. For example, an implant opening may be aligned with a fluid passageway that extends to/from the tissue structure or organ into which it is implanted. For example, the implant may have a structure configured for placement within a portion of the urinary bladder where an opening is provided in the implant for alignment with the urethra. Other examples of organ to lumen junctures within the body that may be treatable with the present invention is the stomach-duodenum juncture, the liver-bile duct juncture, the uterus-fallopian tube juncture, the kidney-renal juncture, the bladder-urethra juncture, etc. Lumen to lumen junctures may also be treated with the present invention. Examples of such include the aorta and any vessel that branches from it, e.g., the coronary ostia, the iliac artery, the subclavian artery, the carotid aretery, the renal arteries, etc. In these later embodiments, one or more openings may be provided in walls of the implant or the implant may have a branched configuration for placement within one or more vessels. On the other hand, the implant site may be more optimally treated with a planar device, such as implant 14 of FIG. 1C, having a hole or fenestration 22 (in phantom) to be aligned with a luminal opening.

The material(s) used to fabricate the subject devices is/are selected to provide a physical or structural function (e.g., buttressing of stenotic vessel) and/or to induce one or more biological (e.g., endothelialization) or therapeutic (via eluted drugs) effects upon the target site at which the device is implanted. Both or only one of the surfaces of the device may be configured to provide a biological or therapeutic effect. Where only one surface is used biologically or therapeutically, the entirety of that surface may be uniformly treated or one or more portions thereof may be so treated. In the latter embodiments, the portions may be similarly or differently treated to impart different biological or therapeutic effects. Where both surfaces of the device (whether in a tubular/hollow configuration or in a planar configuration) are employed biologically or therapeutically, they can be similarly configured, or they may be configured differently where the tissue or environments to which they are each engaged or exposed are different or require different treatments. For example, the exterior surface of a tubular device for treating a stenotic artery may be adapted to induce endothelization at the arterial wall while the interior surface of the device may be adapted to prevent thrombus formation at the implant site. A planar device to be used as a myocardial patch, for example, may have one side, i.e., the cardiac-contacting surface, treated to impart an angiogenic effect on the myocardium, and the other side, i.e., the pericardial facing surface, may be treated to minimize the risk of adhesion or inflammation at the interior portion of the pericardium that comes into contact with the patch. Another application for which the planar embodiments are well suited is the treatment of vascular aneurysms where one side of the patch is treated to seal over the mouth of the aneurysm and the other side is adapted to endothelialize and integrate into the healthy portion of the vascular wall.

The subject devices may also be used to treat other conditions such as cancer, diverticulitis and physical trauma caused to a vessel or organ. For cancer applications, the devices are seeded with chemotherapy eluding drugs. The optimal shape of the implant may vary depending on the location of the tumor. For example, a highly vascularized tumor may be well-suited for using a tubular implant placed in a vessel supply blood to the tumor. Alternatively, a patch or other planar implant may be applied directly to the surface of the tumor. To treat diverticulitis in the intestinal tract, the implant may be tubular or planar, having the appropriate therapeutic agents to be released into the intestinal wall.

The implants are particularly constructed to have dimensions, i.e., diameters, length sand surface areas, to accommodate the target tissue at the host site. In coronary applications, for example, the outer diameter of the tubular implant will typically range from about 2 mm to about 5 mm. Where the implant is designed for use in an organ or at a juncture between an organ and a vessel, the diameter may vary along the implant's length. The length of the implant is selected to address the extent of disease or damage at the implant site; however, where the disease or damage covers an exceptionally lengthy portion of tissue, the area may have to be treated with more than one implant. The tubular implants and planar implants have lengths and surface areas, respectively, that closely match that of the target tissue site so as not to unnecessarily cover healthy tissue.

The implantable device may be constructed of a single layer of material, or include multiple layers of the same or different material. The latter configuration may be beneficial in circumstances where a finely controlled timing of drug release is desired. For example, an outer layer of the implant, i.e., that which is to be placed in direct contact with a surface (whether exterior or interior) of a vessel or organ implant site, may comprise an agent that prepares the tissue surface for healing processes. An inner layer having a different drug will then be in direct contact with the tissue wall once the first or outer layer has dissolved or biodegraded or otherwise exhausted its drug eluting potential. The process is continued for additional layers. A similar process may ensue within the other side/interior of the implant, starting with the exposed layer of the implant. In certain embodiments, the dual processes occur in parallel with each other, where each layer has a selected agent to be released over a selected time period.

Method and apparatus for releasing active substances from implantable and other devices are described in U.S. Pat. Nos. 6,096,070; 5,824,049; 5,624,411; 5,609,629; 5,569,463; 5,447,724; and 5,464,650. The use of hydrocylosiloxane as a rate limiting barrier is described in U.S. Pat. No. 5,463,010. Coatings to enhance biocompatibility of implantable devices are described in U.S. Pat. Nos. 5,463,010; 5,112,457; and 5,067,491. Energy based devices are described in U.S. Pat. Nos. 6,031,375; 5,928,145; 5,735,811; 5,728,062; 5,725,494; 5,409,000, 5,368,557; 5,000,185; and 4,936,281. Magnetic processes, some of which have been used in drug delivery systems, are described in U.S. Pat. Nos. 5,427,767; 5,225,282; 25 5,206,159; 5,069,216; 4,904,479; 4,871,716; 4,501,726; 4,357,259; 4,345,588; and 4,335,094.

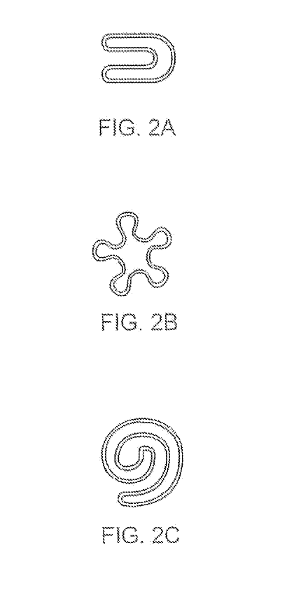

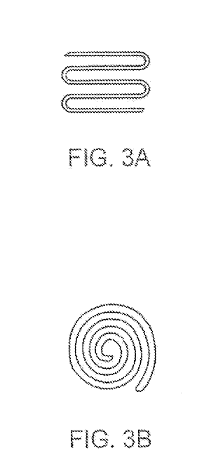

The tubular/hollow implantable devices of the present invention are designed in most cases to have some radial expandability, wherein they are deliverable in a reduced or unexpanded state and then, upon placement at a target site, are caused to expand to engage the walls of the implant site. In other embodiments, the implant is compressible from a natural state to a reduced state for delivery purposes. For example, as illustrate in the cross-sectional views of FIGS. 2A-2C, a tubular implant 24 may be made of a material that is foldable (FIG. 2A), pleated (FIG. 2B) or flattened and rolled upon itself (FIG. 2C), whereby the implant is placed in the reduced profile or diameter state for delivery and then, upon reaching the target site, is allowed or caused to unfold or unroll to the higher profile state. The planar implants, as illustrated in cross-sectional views of FIGS. 3A and 3B, may also be designed to be folded (FIG. 3A) or rolled (FIG. 3B) to a profile that enables delivery through a tubular delivery instrument and/or a small access site, and then unfolded/unrolled when released from the delivery instrument.

In variations in which the implants have a more rigid or less flexible structure, they can be likened to a stent (when tubular) or a substrate (when planar), and act as a scaffold which carries or supports other components, materials, films, agents, cells, etc., as discussed above. In variations in which the implants have a less rigid or more flexible structure, they may be more likened to a graft which may or may not require either the temporary or permanent engagement with another structure, such as a conventional stent or the like, for support during delivery and/or subsequent to placement at the target site. In the latter variation, the implant may be likened to a stent-graft. With any of the stent embodiments, conventional stent materials such as stainless steel, elgiloy, tungsten, platinum or nitinol as well as any other suitable materials may be used instead of or in addition to these commonly used materials.

The use of stents for drug delivery within the vasculature is described in PCT Publication No. WO 01/01957 and U.S. Pat. Nos. 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,769,883; 5,735,811; 5,700,286; 5,679,400; 5,649,977; 5,637,113; 5,591,227; 5,551,954; 5,545,208; 5,500,013; 5,464,450; 5,419,760; 5,411,550; 5,342,348; 5,286,254; and 5,163,952. Methods for coating of stents are described in U.S. Pat. No. 5,356,433.

Given the known drawbacks of commonly used implants, such as metal stents and the like, the present invention contemplates forming the subject implantable devices, at least in part, from biological materials. Preferable biological materials are those which are resorbable by the body and are able to impart a biological or therapeutic effect on the tissue at the implant site. As such, suitable materials include, but are not limited to, extracellular matrices (ECMs), acellularized uterine wall, decellularized sinus cavity liner or membrane, acellular ureture membrane, umbilical cord tissue, decelluarized pericardium and collagen. Other biodegradable materials are described in U.S. Pat. Nos. 6,051,276; 5,879,808; 5,876,452; 5,656,297; 5,543,158; 5,484,584; 5,176,907; 4,894,231; 4,897,268; 4,883,666; 4,832,686; and 3,976,071.

The ECM materials may be natural or synthetic. Natural ECM materials suitable for use with the present invention include mammalian small intestine submucosa (SIS), stomach submucosa (SS), urinary bladder submucosa (UBS), dermis, or liver basement membranes (LBM) derived from sheep, bovine, porcine or any suitable mammal. Small intestine submucosa (SIS) is described in U.S. Pat. Nos. 4,902,508 (hereinafter the '508 patent), 4,956,178 (hereinafter the '178 patent) and 5,275,826; urinary bladder submucosa (UBS) is described in U.S. Pat. No. 5,554,389 (hereinafter the '389 patent), stomach submucosa (SS) is described in U.S. Pat. No. 6,099,567, and liver submucosa (LS) or liver basement membrane (LBM) is described in U.S. Pat. No. 6,379,710, the disclosures of which are incorporated herein by reference. Extracellular matrix-like materials are also generally described in the article “From Cell-ECM Interactions to Tissue Engineering”, Rosso et al, Journal of Cellular Physiology 199.174-180 (2004).

Native extracellular matrices are prepared with care that their bioactivity for tissue regeneration is preserved to the greatest extent possible. Key functions that may need to be preserved include control or initiation of cell adhesion, cell migration, cell differentiation, cell proliferation, cell death (apoptosis), stimulation of angiogenesis, proteolytic activity, enzymatic activity, cell motility, protein and cell modulation, activation of transcriptional events, provision for translation events, inhibition of some bioactivities, for example inhibition of coagulation, stem cell attraction, and chemotaxis. Assays for determining these activities are standard in the art. For example, material analysis can be used to identify the molecules present in the material composition. Also, in vitro cell adhesion tests can be conducted to make sure that the fabric or composition is capable of cell adhesion.

Many of these ECM compositions are generally comprised of the same tissue layers and are prepared by the same method, the difference being that the starting material is small intestine on the one hand and urinary bladder on the other. The matrices are generally decellularized in order to render them non-immunogenic. A critical aspect of the decellularization process is that the process be completed with some of the key protein function retained, either by replacement of proteins incidentally extracted with the cells, or by adding exogenous cells to the matrix composition after cell extraction, which cells produce or carry proteins needed for the function of tissue regeneration in vivo. Specific procedural steps are further detailed in the patents referenced above. For example, the '508, '389 and '178 patents, disclose mechanical abrading steps to remove the inner layers of the tissue, including at least the lumenal portion of the tunica mucosa of the intestine or bladder, i.e., the lamina epithelialis mucosa (epithelium) and lamina propria. Abrasion, peeling, or scraping the mucosa delaminates the epithelial cells and their associated basement membrane, and most of the lamina propria, at least to the level of a layer of organized dense connective tissue, the stratum compactum. Thus, the ECMs previously recognized as soft tissue replacement material is devoid of epithelial basement membrane and consists of the submucosa and stratum compactum.

Examples of a typical epithelium having a basement membrane include, but are not limited to the following: the epithelium of the skin, intestine, urinary bladder, esophagus, stomach, cornea, and liver. The epithelial basement membrane may be in the form of a thin sheet of extracellular material contiguous with the basilar aspect of epithelial cells. Sheets of aggregated epithelial cells of similar type form an epithelium. Epithelial cells and their associated epithelial basement membrane may be positioned on the luminal portion of the tunica mucosa and constitute the internal surface of tubular and hollow organs and tissues of the body. Connective tissues and the submucosa, for example, are positioned on the abluminal or deep side of the basement membrane. Examples of connective tissues used to form the ECMs that are positioned on the abluminal side of the epithelial basement membrane include the submucosa of the intestine (SIS) and urinary bladder (UBS), and the dermis and subcutaneous tissues of the skin. The submucosa tissue may have a thickness of about 80 micrometers, and consist primarily (greater than 98%) of a cellular, eosinophilic staining (H&E stain) extracellular matrix material. Occasional blood vessels and spindle cells consistent with fibrocytes may be scattered randomly throughout the tissue. Typically the material is rinsed with saline and optionally stored in a frozen hydrated state until used.

In addition to employing intact ECMs to form the devices of the present invention, the ECM material may be fluidized or emulsified and mixed or extruded with or placed or wrapped around another structure. Fluidized UBS, for example, can be prepared in a manner similar to the preparation of fluidized intestinal submucosa, as described in U.S. Pat. No. 5,275,826, the disclosure of which is expressly incorporated herein by reference. The UBS is comminuted by tearing, cutting, grinding, shearing or the like. Grinding the UBS in a frozen or freeze-dried state is preferred although good results can be obtained as well by subjecting a suspension of submucosa pieces to treatment in a high speed (high shear) blender and dewatering, if necessary, by centrifuging and decanting excess water. Additionally, the comminuted fluidized tissue can be solubilized by enzymatic digestion of the bladder submucosa with a protease, such as trypsin or pepsin, or other appropriate enzymes for a period of time sufficient to solubilize said tissue and form a substantially homogeneous solution.

Powder forms of ECMs may also be used to coat other materials used to form the subject implants. In one embodiment a powder form of UBS is prepared by pulverizing urinary bladder submucosa tissue under liquid nitrogen to produce particles ranging in size from 0.1 mm to 1 mm². The particulate composition is then lyophilized overnight and sterilized to form a solid substantially anhydrous particulate composite. Alternatively, a powder form of UBS can be formed from fluidized UBS by drying the suspensions or solutions of comminuted UBS.

Other examples of ECM material suitable for use with the present invention include but are not limited to dermal extracellular matrix material, subcutaneous extracellular matrix material, large intestine extracellular matrix material, placental extracellular matrix material, ornamentum extracellular matrix material, heart extracellular matrix material, and lung extracellular matrix material, may be used, derived and preserved similarly as described herein for the SIS, SS, LBM, and UBM materials. Other organ tissue sources of basement membrane for use in accordance with this invention include spleen, lymph nodes, salivary glands, prostate, pancreas and other secreting glands. In general, any tissue of a mammal that has an extracellular matrix can be used for developing an extracellular matrix component of the invention.

Other materials can be used to synthesize ECMs. These include but are not limited to fibronectin, fibrin, fibrinogen, collagen, including fibrillar and non-fibrillar collagen, adhesive glycoproteins, proteoglycans, hyaluronan, secreted protein acidic and rich in cysteine (SPARC), thrombospondins, tenacin, cell adhesion molecules, and matrix metalloproteinase inhibitors.

When using collagen-based synthetic extracellular matrix materials, the collagenous matrix can be selected from a variety of commercially available collagen matrices or can be prepared from a wide variety of natural sources of collagen. Collagenous matrix for use in accordance with the present invention comprises highly conserved collagens, glycoproteins, proteoglycans, and glycosaminoglycans in their natural configuration and natural concentration. Collagens can be from animal sources, from plant sources, or from synthetic sources, all of which are available and standard in the art. In addition, collagen from mammalian sources can be retrieved from matrix containing tissues and used to form a matrix composition.

Synthetic extracellular matrices can also be formed using synthetic molecules that polymerize much like native collagen and which form a scaffold environment that mimics the native environment of mammalian extracellular matrix scaffolds. Materials such as polyethylene terephthalate fiber (Dacron), polytetrafluoroethylene (PTFE), glutaraldehyde-cross linked pericardium, polylactate (PLA), polyglycol (PGA), hyaluronic acid, polyethylene glycol (PEG), polyethelene, nitinol, and collagen from non-animal sources (such as plants or synthetic collagens) can be used as components of a synthetic extracellular matrix scaffold. The synthetic materials listed are standard in the art, and forming hydrogels and matrix-like materials with them is also standard. Their effectiveness can be tested in vivo as sited earlier, by testing in mammals, along with components that typically constitute native extracellular matrices, particularly the growth factors and cells responsive to them.

The subject implantable devices may also be fabricated from a combination of materials, for example, an extracellular matrix component and a polymeric material where the latter is formed as a scaffold to which the ECM material is applied or adhered. Particularly useful polymers are those which are biodegradable and/or bioabsorbable. These include but are not limited to polylactides, poly-glycolides, polycarprolactone, polydioxane and their random and block copolymers. Examples of specific polymers include poly D,L-lactide, polylactide-co-glycolide (85:15) and polylactide-co-glycolide (75:25). Preferably, the biodegradable and/or bioabsorbable polymers used in the fibrous matrix of the present invention will have a molecular weight in the range of about 1,000 to about 8,000,000 g/mole, more preferably about 4,000 to about 250,000 g/mole. Examples of suitable polymers can also be found in Bezwada, Rao S. et al. (1997) Poly(p-Dioxanone) and its copolymers and in the Handbook of Biodegradable Polymers, A. J. Domb, J. Kost and D. M. Wiseman, editors, Hardwood Academic Publishers, The Netherlands, pp. 29-61.

The biodegradable and/or bioabsorbable polymer may contain a monomer selected from the group consisting of a glycolide, lactide, dioxanone, caprolactone, trimethylene carbonate, ethylene glycol and lysine. The material can be a random copolymer, block copolymer or blend of monomers, homopolymers, copolymers, and/or heteropolymers that contain these monomers. The biodegradable and/or bioabsorbable polymers may also contain bioabsorbable and biodegradable linear aliphatic polyesters such as polyglycolide (PGA) and its random copolymer poly(glycolide-co-lactide-) (PGA-co-PLA). The FDA has approved these polymers for use in surgical applications, including medical sutures. An advantage of these synthetic absorbable materials is their degradability by simple hydrolysis of the ester backbone in aqueous environments, such as body fluids. The degradation products are ultimately metabolized to carbon dioxide and water or can be excreted via the kidneys. These polymers are very different from cellulose based materials, which cannot be absorbed by the body.

Other examples of suitable biocompatible polymers are polyhydroxyalkyl methacrylates including ethylmethacrylate, and hydrogels such as polyvinylpyr-rolidone, polyacrylamides, etc. Other suitable bioabsorbable materials are biopolymers which include collagen, gelatin, alginic acid, chitin, chitosan, fibrin, hyaluronic acid, dextran, polyamino acids, polylysine and copolymers of these materials. Any glycosaminoglycan (GAG) type polymer can be used. GAGs can include, e.g., heparin, chondroitin sulfate A or B, and hyaluronic acid, or their synthetic analogues. Any combination, copolymer, polymer or blend thereof of the above examples is contemplated for use according to the present invention.

In addition to the structural and biological functions provided by the subject implants, the implants may be fabricated with materials which are capable of releasing one or more therapeutic agents at the target site in a controlled manner, e.g., eluting a drug that inhibits restenosis or hyperplasia. Materials suitable for this purpose include but are not limited to poly-1-lactic acid/poly-ε-caprolactone copolymer, polyanhydrides, polyorthoesters, polycaprolactone, poly vinyl acetate, polyhydroxybutyrate/polyhyroxyvalerate copolymer, polyglycolic acid, polyactic/polyglycolic acid copolymers and other aliphatic polyesters, among a wide variety of polymeric substrates available for devices that can be placed in a human body.

Another feature of the present invention is that, in certain embodiments, the subject implants are designed to be carried at a distal portion of a delivery instrument, such as a catheter or the like, and released therefrom. This may enable use of a smaller diameter delivery instrument or catheter than would otherwise be required if the device were to be preloaded therein. In these embodiments, the catheter may be characterized as a pusher against which the proximal end of the implant abuts, thereby enabled to be pushed through the passageway to the target site, i.e., rather than being carried within the catheter.

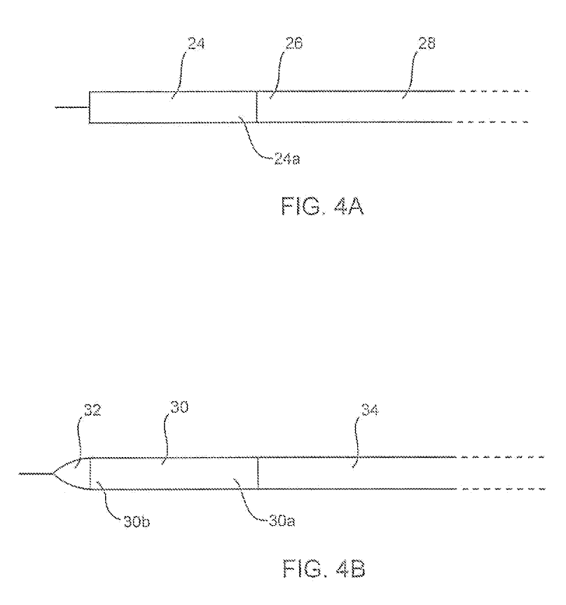

The particular location of the implant relative to the delivery instrument when operatively loaded or attached thereto may vary according to the application in which it is being used. In one variation, as illustrated in FIG. 4A, the implant 24 is positioned and carried distally of the very distal end 26 of the delivery device 28 whereby the implant's proximal end 24a is releasably attached to the delivery device and the implant's distal end 24b is unattached. In another variation, as illustrated in FIG. 4B, the implant 30 is carried proximal to the distal tip 32 of the delivery catheter 34, for example, with the use of a dilator or nose cone 32. In this embodiment, either the implant's proximal end 30a or distal end 30b or both may be releasably attached to the delivery device/distal tip.

In certain variations, the implants are fabricated separately from the delivery instrument and are physically attached or secured in a releasable manner thereto, such as by way of one or a plurality of attachment/release mechanisms, e.g., sutures, strings, magnets, clips, hooks, etc. The attachment-release mechanisms may be designed to remain with the delivery instrument, the implant or both, or otherwise be designed to detach from both the implant and the delivery instrument. Where they are to remain with the implant, the mechanisms may be made of biodegradable or instantly dissolvable materials. However, in vascular applications, this arrangement is not advisable due to the risk of blockage, embolism and thrombus formation. Obviating this concern, a bio-adhesive may be used to secure the implant to the end of the delivery catheter, where the adhesive material has properties which enable it to dissipate or dissolve when exposed to moisture and/or body heat at the target tissue site.

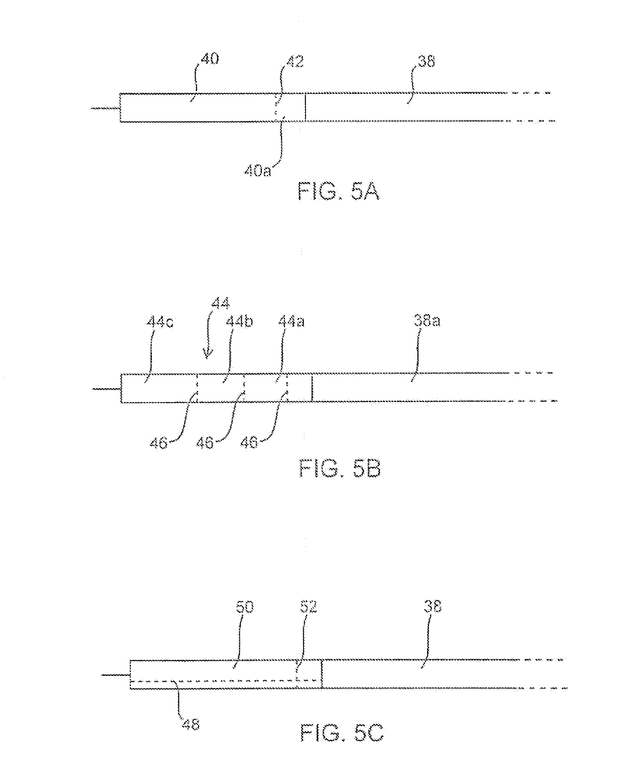

In other variations, the implant or a portion thereof may be fabricated or integrated as part of the delivery instrument and configured to be separated from the delivery instrument only upon placement at the implant site. For example, as illustrated in FIG. 5A, the implant 40 or a portion thereof may be separable or released from the delivery device 38 by way of perforations 42 provided in the material which can be caused to split or tear away from the delivery device 38. The portion 40a of the implant which is to remain with the delivery instrument 38, as the case may be, may be secured to the delivery instrument by a permanent adhesive or one that does not loosen or dissolve when exposed to moisture and/or heat.

An implant 44, as illustrated in FIG. 5B, having an extended length may be provided with multiple sets 46 of perforations about its circumference at spaced apart locations along its length in order to allow the user to select the appropriate length or portion of the device to be released. With this variation, a couple of complications and inconveniences can be avoided. First, by having the flexibility to employ a longer implant device, the need to separately deliver two or more implants to cover an extensively diseased or damaged tissue area is obviated. Further, where the affected target area is particularly small or short, applying the implant unnecessarily to healthy tissue is avoided. Additionally, with multiple perforation sets, two or more implant portions or segments may be separately released from the same delivery instrument during the same procedure to cover spaced apart target sites. This variation is particularly useful, for example, in treating a blood vessel with multiple stenotic lesions along its length. In such an application, the distal most perforated segment 44c of the implantable tubular device is advanced to the most distal implant site and then released. The implant is then retracted, if necessary, such that the newly disposed distal segment 44b is positioned at a more proximal implant site. These steps may be repeated (for segment 44a and so on) as necessary up to a number of repetitions that equals that of the number of implant segments provided.

In another variation, as illustrated in FIG. 5C, the perforations 48 may be placed in a linear fashion within the implant material 50. For tubular implants, this means that the perforations run substantially along the longitudinal axis of the implant, although additional circumferential perforations 52 may be provided to facilitate release of the implant from the delivery device 38. For more spherically shaped hollow implants, the perforations run substantially parallel to the major axis of the implant (although the perforations may be aligned with the minor axis, or both). In either case, the implants, when expanded, are caused to split lengthwise with the unrestrained resulting structure being a planar sheet or strip. As such, the implants may be delivered in a shape suitable for translation through a catheter, and thereafter, upon release from the catheter, be transformed to another configuration more suitable for the tissue surface to be treated. This variation is ideal for enabling minimally invasive, e.g., percutaneous, delivery of a sheet or patch device to a target site which, in its planar form, would not otherwise be deliverable in a minimally invasive manner. Exemplary applications for this variation include delivery of a sheet or patch implant in the initial form of a longitudinally perforated tube to the myocardium through a thorascopic access site, through a laproscopic access site to repair a hernia in need of repair, through the urethra to treat the bladder, etc.

The source and type of force needed to cause the perforations within the implant material to separate may also vary. In one variation, the force is sourced within the interior of the implant's lumen and radially applied to the implant. In another variation, a linearly directed tension or pulling force is employed to separate an implant's perforations. The force may be applied to one or both ends of the implant in a direction away from the attachment point.

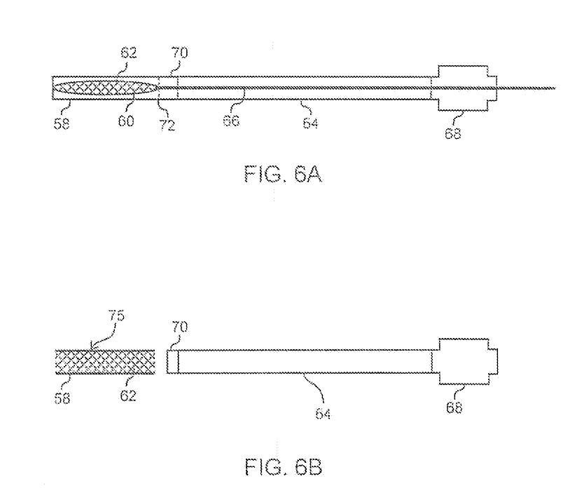

In those embodiments employing radial force, such force may be applied to the implant by the expansion of an expandable member carried by or associated with the delivery-placement system and positioned within the interior of the implant. As illustrated in FIGS. 6A and 6B, the expandable member 60 may be a balloon, mesh or the like, which is delivered within a reduced or unexpanded state during delivery (see FIG. 6A) to the implant site, and then caused to expand (by inflation or mechanical means) when ready to place the device 58 at the desired site. In embodiments where the implant structure is to include a stent 62 or stent-like component over which the biological material is placed, the expandable member 60 acts directly upon the stent 62 in much the same way balloon-expandable stents are placed in conventional procedures.

Various steps or acts involved in using the system of FIGS. 6A and 6B are now described in more detail FIG. 6A illustrates a catheter apparatus 64 having a balloon catheter 66 disposed therein. Stent 62 has been operatively placed or loaded about balloon member 60 which is disposed within device 58, and depicted in the figure in a partially inflated condition. Functional operation of catheter apparatus 64 may be conducted from a luer fitting 68 positioned at a proximal end of catheter 64. Balloon catheter 66 may be introduced over a guidewire (not shown) or dilator (also not shown) in order to position stent-mounted balloon 60 within device 58. Upon inflation of balloon 60, stent 62 is caused to expand radial outwards to engage the interior wall of tubular implant 58. Upon further expansion of the stent 62, the radial force applied to device 58 causes the perforations 72 or attachments mechanisms (not shown) to break loose or split apart from the distal end 70 of catheter 64, as illustrated in FIG. 6B. With stent 62 fully deployed and engaged within device 58, device 58 is pressed against the luminal wall at the implant site (not shown), and held there by the interior pressure provided by stent 62. As such, a unified tubular implant 75 is provided and positioned at the target tissue site.

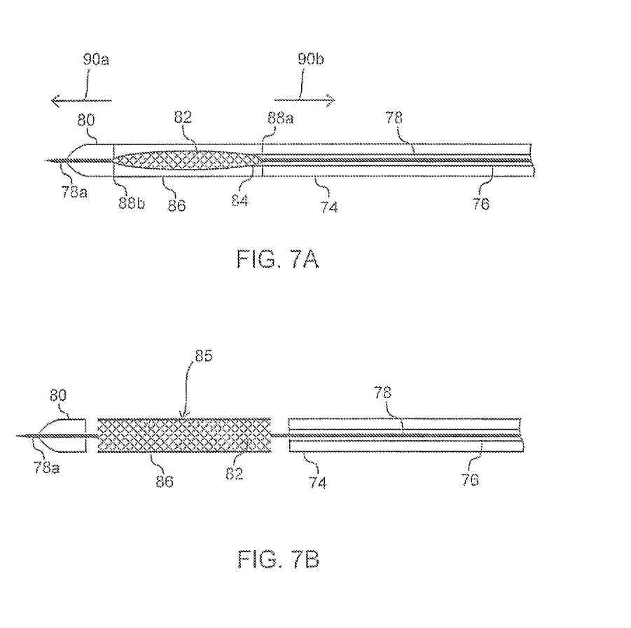

FIGS. 7A and 7B illustrate a variation of the system just described with the additional use of a guidewire 78 having a nose cone 80 disposed at its distal end 78a. Catheter apparatus 74 is provided with implantable device 86 is releasably attached at its proximal end to the distal end of catheter lumen 74 and at its distal end to the proximal side of nose cone 80. Once catheter 74 with guidewire 78 have been delivered to the target site, balloon catheter 76 may be introduced over the guide wire through catheter lumen 74 in order to position stent-mounted balloon 84 within device 86. The radial force applied to device 58 by the expansion of stent 82 causes the perforations 88a and 88b or attachments mechanisms (not shown) to break loose or split apart from the distal end of catheter 64 and the proximal side of nose cone 80, as illustrated in FIG. 7B. Once the unified tubular implant 85 is positioned at the target tissue site, guide wire 78, along with balloon catheter 76, are retracted whereby nose cone 80, having a smaller diameter than inner diameter of the fully deployed implant 85, passes within implant 85.

Alternatively, separation of the implant 86 from the delivery system may be accomplished by applying tension (with or without the application of radial force) to the device by manipulating components of the delivery system. This may be accomplished in a variety of ways. Guidewire 78 may be advanced in a distal direction such that the attached nose cone 80 pulls the implant in a distal direction 90a while catheter body 74 is held stationary. Alternatively, guidewire 78 and nose cone 80 may be held stationary while catheter body 74 is pulled in a proximal direction 90b thereby placing implant 86 in tension. Still yet, the respective pulling actions may be applied simultaneously. In either case, the applied tension causes perforations 88a and 88b to split thereby releasing implant 82 at both ends from the delivery system.

In other variations (not illustrated), the implants are self-expanding where the radial force is inherent or stored in the implant's structure. Self-expanding stents are well known in the art and may be used with non-stent materials forming the implant, or the self-expanding features may be incorporated into a non-stent component of the implant thereby obviating the need for a stent. For example, polymeric materials may be specifically fabricated to provide a resiliency to the implant whereby a radial spring force is provided by the implant when the implant is compressed, folded, rolled, pleated, etc. A sleeve or the like may be employed over the self-expanding implant to maintain its reduced state during delivery to the implant site and then removed (by retracting or opening the sleeve) to deploy the implant at the site. Where the self-expanding implant is restrained by direct attachment to the delivery system, as described above, the perforations, strings or the like, may be cut or severed by means of a cutting instrument incorporated into the delivery system. Such instrument may provide a radial blade which is rotationally moveable, radially expandable (if positioned on the interior of the implant) or radially compressible (if positioned about the exterior of the implant). A straight blade aligned along the longitudinal axis of the catheter may be used whether the perforations, strings, seems or the like to be cut run longitudinally along the implant.

A particular procedure for placing a tubular implant including a stent within a vessel of a living body is now particularly described. Typically, a standard guidewire is advanced into the vessel lumen across the lesion of interest with sufficient room to place a stent. A delivery catheter having the tubular implant (still attached, but detachable) is advanced over the guidewire to place it at the lesion site. A stent catheter carrying a stent (the stent can be, e.g., either alone and self expanding or disposed over a balloon) is then back-loaded over the guidewire but disposed within the delivery catheter and advanced to the lesion inside the tube. The stent is expanded, e.g. either by inflation of a balloon, or by a self-expanding means intrinsic to or within the stent, e.g., a spring-like capability in the stent, and thereby contacts the interior wall of the tubular implant. As the stent continues to expand, the detachable implant expands radially with the stent, and then becomes trapped or sandwiched between the stent outer diameter and the inner diameter or surface of the vessel lumen. During the expansion sequence, the perforations or attachments around the circumference of the implant will tear and yield therefore providing for the detachment of the implant from the catheter.

After confirming detachment of the expanded tubular implant with the stent, the stent balloon is deflated and withdrawn from the catheter shaft. If another mechanism other than an expanding balloon is used, then that expanding and delivery mechanism is likewise withdrawn. After the stent catheter is removed, the implant delivery catheter is removed. Correct sizing of the implant and stent lengths is taken into consideration in order that they match the length of the lesion or blocked area in the vessel lumen. The stent diameter size is also important in order that the stent contact and exert adequate pressure on the interior of the implantable tube to fully expanded the tube and maintain that expansion to the point of contact of the lumen wall.

A primary advantage of a tubular structure disposed in contact with a stent is that the tube can be used with any commonly manufactured stent. Additionally, the usually rigorous processing of a drug eluting stent is obviated because there is not coating required for the stent and thus the present invention can employ less costly bare metal stents in lieu of drug eluting stents. The detachable tube will perform the function of delivering drug to the site of defect in the lumen while the stent that expands within it and holds it in place against the lumen wall will provide support architecture at the site of defect. If the detachable tube is made of extracellular matrix material, the therapeutic nature of the extracellular matrix material as it remodels into adjacent healthy parent tissue may restore the lumen to an original healthy state, while the remaining stent will maintain supporting architecture for the healing tissue.

Construction of the implantable devices of the invention is accomplished by standard catheter construction with regard to the implant delivery catheter and, if a stent is employed, to the stent delivery catheter. For example, the luer and catheter shafts are constructed using conventional techniques typically used in the manufacture of catheter products. The catheter shaft can be a single lumen extruded polymer affixed with a conventional luer. The inner diameter of the catheter shaft should be capable of receiving and allowing free movement of a commercialized stent/balloon catheter along its entire length. The detachable tube can be fixed to the catheter shaft using conventional techniques like adhesives, heat shrink tubing, sewing, overmolding and the like. The detachable tube can be attached to the inner or outer diameter of the catheter shaft at spaced apart intervals (e.g., by providing perforations) sufficient to allow for the detachment of the tubular implant by expansion of the stent via balloon inflation or by the spring force of a self-expanding stent. Another means of detachment of the tube is allowing for the detachment by way of radial force imparted on the detachable tube sufficient enough to overcome the fixing means. For example, the detachment could be accomplished by overcoming the adhesive forces of the fixing adhesive, detachment by radial expansion greater than the radial force imparted by the heat shrink tubing, and by tearing or yielding of the detachable tube material or threads used to affix the detachable tip to the catheter shaft.

There are many ways to construct the implantable materials in whatever configuration (e.g., tubular, hollow, planar, etc) desired. The implants may be formed using a sheet of material, for example extracellular matrix or other therapeutic material prepared as described above, then rolled into a tube where the two opposing or overlapping edges can be sewn together using conventional practices, for either permanent engagement (i.e., for tubular implants), temporary engagement (i.e., for planar implants or portions of permanent attachment and portions of temporary attachment (i.e., for implants having both tubular and planar portions). Alternatively, tubular implants may be extruded as a tube wherein the material can be forced through an opening provided by the extruding internal shape (for example a rod or mandrel) and the extruding external shape (for example a ring or dye head). Alternatively, the implants may be shaped for example by dipping, spraying or electrostatic processes wherein the material is a fluid, gel, powder, or emulsification capable of adhering to a mold shape. The material is then formed or wrapped around a mandrel or mold and, after processing, is then removed therefrom having the desired shape/configuration.

The implant material may be selected to biodegrade over a desired time period after placement within the body. Where the implant comprises extracellular matrix, the matrix material can promote healing and generation of healthy tissue at the site of defect. The implant may comprise other biodegradable materials and may also comprise drug-containing or drug-eluting materials. The drugs that may be placed or incorporated within the materials include any drug believed to be efficacious in treatment of a defect or in prevention of a condition, including any drug having an in vivo release profile compatible with the goals of the treatment. Commonly used drugs in vascular applications include those which promote endothelization of a luminal wall, and anti-thrombotic drugs to prevent blockage by drug clot formation in the lumen and elsewhere in the body. Anti-proliferative drugs may also be used to prevent restenosis in vascular lumens. Other drugs appropriate for the particular treatment objectives may also be used. The implant material(s) (e.g., where the tube is layered using more than one material or is itself a combination of materials) may also present more than one drug, e.g., where the drugs can work in concert, or where each administered drug is directed to a different but compatible therapeutic objective at the site of defect or in the body generally. An implant comprised of more than one layer of material can present a different drug to the body in each layer.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

It must be noted that as used herein and in the appended claims, the singular forms “a”, “an”, and “the” include plural referents unless the context clearly dictates otherwise. Thus, for example, reference to “an releasable attachment mechanism” may include a plurality of such mechanisms, and reference to “the stent” includes reference to one or more stents and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

What is claimed is:

1. An implantable device comprising a biocompatible material releasably attachable to a delivery instrument for placement within in the body, wherein the material comprises perforations therein.

2. The device of claim 1, wherein the material comprises perforations at a proximal end of the device configured for releasable attachment to the delivery instrument.

3. The device of claim 1, wherein the material comprises perforations at a distal end of the device configured for releasable attachment to the delivery instrument.

4. The device of claim 1, wherein the material comprises perforations at a proximal end and a distal end of the device configured for releasable attachment to the delivery instrument.

5. The device of claim 1, wherein the device is releasable from the delivery instrument by breaking the perforations.

6. The device of claim 1, wherein the device has a tubular, hollow or planar configuration.

7. The device of claim 1, wherein the device is self-expandable upon release from the delivery instrument.

8. The device of claim 1, wherein the device is expandable upon application of a radial force on an interior surface of the device.

9. The device of claim 1, wherein the perforations are circumferentially placed about the device when releasably attached to the delivery device.

10. The device of claim 1, wherein the perforations are in linear alignment with the longitudinal axis of the delivery instrument.

11. The device of claim 1, wherein the material is biodegradable or bioresorbable.

12. The device of claim 1, wherein the material further comprises a therapeutic agent which is eludable from the material.

13. The device of claim 1, wherein the material comprises an extracellular matrix.

14. A system for treating a defect at a target tissue site within the body, the system comprising:
a catheter; and
an implantable material releasably attached at a distal portion of the catheter, wherein the material comprises perforations therein.

15. The system of claim 14, wherein the implantable material has a tubular configuration when releasably attached to the catheter.

16. The system of claim 15, further comprising a mechanism positioned within the tubular material for exerting a radial force on the tubular material sufficient to separate the perforations.

17. The system of claim 16, wherein the perforations are positioned circumferentially about the tubular material.

18. The system of claim 16, wherein the perforations are positioned longitudinally along the tubular material.

19. The system of claim 16, wherein the mechanism is an inflatable balloon or an expandable mesh.

20. The system of claim 15, wherein the material retains a tubular configuration when released from the catheter.

21. The system of claim 15, wherein the material has a planar configuration when released from the catheter.

22. A method of making a device for delivery to a body lumen, the device comprising a material which is configured to induce a biological or therapeutic effect when placed at tissue site within the body, the method comprising:
forming the material in the desired shape and having the desired dimensions;
applying perforations in the material, and
attaching the material to a catheter for delivery to within the body, wherein the perforations are arranged relative to the catheter such that breaking the perforations releases the device from the catheter.

23. The method of claim 22, wherein forming the material comprises a process selected from the group consisting of extruding, sewing, laminating, pressing, freeze-drying, gluing, and molding.

24. The method of claim 22, wherein the material is selected from the group consisting of extracellular matrix derived from a mammal, synthetic extracellular matrix, an extruded material, a biodegradable material, and a drug eluting material.

25. A method of treating a tissue site within the body, comprising:
releasing a device in the vessel lumen of a living body from a distal portion of a catheter by breaking perforations provided in the device, wherein the device comprises a material for inducing a biological or therapeutic effect at the tissue site.

26. The method of claim 25, wherein the material contains at least one therapeutic agent and the method further comprises eluding the at least one therapeutic agent from the material.

27. The method of claim 26, wherein the material comprises multiple layers, each containing at least one therapeutic agent, wherein the method further comprises eluding the therapeutic agents sequentially.

28. The method of claim 25, further comprising buttressing the vessel lumen with the released device.
